(19) United States
(12) Patent Application Publication
(10) Pub. No.: US 2016/0101050 A1
(43) Pub. Date: Apr. 14, 2016

(54) OPHTHALMIC NANOEMULSION COMPOSITION CONTAINING CYCLOSPORINE AND METHOD FOR PREPARING SAME

(71) Applicant: TAEJOON PHARM. CO., LTD., Seoul (KR)

(72) Inventors: Joon Youb LEE, Seoul (KR); Youn Jae SHIN, Seoul (KR); Sang-Rok RYU, Gyeonggi-do (KR)

(21) Appl. No.: 14/892,535

(22) PCT Filed: May 20, 2014
(86) PCT No.: PCT/KR2014/004489

§ 371 Date: Nov. 19, 2015

(30) Foreign Application Priority Data
May 20, 2013 (KR) 10-2013-0056561
Mar. 13, 2014 (KR) 10-2014-0029939

(51) Int. Cl. A61K 9/107; A61K 38/13; A61K 47/10; A61K 47/14; A61K 47/26; A61K 9/00; A61K 47/44

(52) U.S. Cl. CPC A61K 9/1075; A61K 9/0048; A61K 38/13; A61K 47/44; A61K 47/14; A61K 47/26; A61K 47/10

(57) ABSTRACT

Provided is an ophthalmic nanoemulsion composition and a method for preparing the same which increases the solubility of cyclosporine as an active ingredient and improves the stability of the ophthalmic composition by mixing cyclosporine, a nonaqueous solvent, an emulsifier, and an aqueous solvent, and a method for preventing or treating dry eye syndrome using the same. The ophthalmic nanoemulsion composition is characterized by having an average particle size of 200 nm or less, preferably 100 nm or less, and having a very narrow particle distribution. Therefore, sterilizing filtration is available, stability is improved, and the composition has superior effect in clinical treatment of dry eye syndrome while minimizing foreign body sensation and visual disturbance, and thus the ophthalmic nanoemulsion composition of the present disclosure can be effectively used as an ophthalmic composition.

### TECHNICAL FIELD

The present disclosure relates to an ophthalmic nanoemulsion composition and a method for preparing the same which increase the solubility of cyclosporine as an active ingredient and improves the stability of the ophthalmic composition by mixing cyclosporine, a nonaqueous solvent, an emulsifier, and an aqueous solvent, and a method for preventing or treating ocular disease using the same.

### BACKGROUND ART

An immunosuppressive drug is a medicine used for immunosuppressive therapy by preventing or inhibiting abnormal immunoreactivity. The immunosuppressive drug is currently being used as a therapeutic agent for various diseases including transplant rejection after the organ and tissue transplantation; inflammatory intestinal disease such as ulcerative colitis or Crohn's disease; rheumatoid arthritis; Behcets syndrome; inflammatory or allergic dermatosis such as psoriasis or atopic dermatitis; inflammatory or allergic respiratory disease such as chronic obstructive pulmonary disease or Asthma; systemic lupus erythematosus; scleroderma; Sjogren syndrome; and dry eye syndrome among others.

Sjogren syndrome is a chronic inflammatory disease of exocrine gland and specifically, it is characterized in decrement of generation of saliva and tear via destruction of normal tissues of salivary gland and lacrimal gland. The cause of Sjogren syndrome is not completely revealed but genetic factors such as family history, viruses, cytokines and autoimmune antibodies are reported as the causes of Sjogren syndrome. Currently, cyclosporine which is an immunosuppressive drug, is used for treating Sjogren syndrome, and nonsteroidal anti-inflammatory drugs or steroids are used along with cyclosporine when symptom is severe.

Dry eye syndrome or immune keratoconjunctivitis sicca (KCS) patients generally complain of soreness, dryness, foreign body sensation and stinging sensation in the eyes. Additionally, it is uneasy to open eyes because eyes are easily being fatigue, and thus feel comfortable when the eyes are closed and symptoms being severe when the eyes are open. The eyes were slightly bloodshot in outward appearance and patients complain headache when symptom is severe. This dry eye syndrome occurs due to tear deficiency caused by insufficient generation and excessive evaporation of tears, imbalance of components of tears, or inflammation of the eye or damage on the intraocular epithelial cells. Within current drugs, a dry eye syndrome supplement (e.g. artificial tears) that temporarily improves dryness and foreign body sensation and cyclosporine as a therapeutic agent that increases tear secretion through immunosuppressive effect in the dry eye are representative.

Cyclosporines are polypeptides consisted of 11 amino acids, and exhibit powerful immunosuppressing activity by inhibiting proliferation and differentiation of T-cell. U.S. Pat. No. 4,839,342 discloses cyclosporine's immunosuppressing activity as well as disclosing that cyclosporines are effective drugs in treating immune keratoconjunctivitis sicca (KCS). Sirolimus, tacrolimus and its derivatives other than cyclosporines are known as ophthalmic preparations.

Cyclosporines have cyclic structure comprising seven N-methylated amino acids and four non-N-methylated amino acids, and there are cyclosporine A, cyclosporine B, cyclosporine C, cyclosporine D and cyclosporine G among others according to the structure of the constituting amino acid residue, and practically, cyclosporine A, of which pharmacological activity and clinical cases are revealed more than the rest, is studied most widely. Intramolecular attraction of cyclosporines is strong and interaction with water molecule is relatively difficult, and thus cyclosporines are poorly water-soluble drugs that are hardly dissolved in water. Water solubility of cyclosporines is known as about 20 g/ml to 30 μg/ml, and it is very difficult to prepare a water-soluble medicament composition with cyclosporine having such low water solubility.

Restasis™, currently being sold as a cyclosporine ophthalmic composition, is a milky-colored opaque emulsion and has demerits of inducement of burning sensation accompanied by conjunctival injection, pruritus, blurred vision and foreign body sensation when it is administered to the eyes. Therefore, a purpose of designing emulsion-type ophthalmic preparation containing the poorly water-soluble cyclosporine as an active ingredient is to stably improve the water solubility of cyclosporine and to minimize the unpleasant symptoms of ophthalmic administration by improving irritation, foreign body sensation, burning sensation, soreness, hyperemia, blurred vision and pruritus.

Existing ophthalmic emulsions generally contain 2 or more immiscible ingredients in a single composition, and thus it is common to form 2 separate phases in a composition.

Thermodynamically, the emulsion is in unstable status and tends to separate into various phases through pathways such as flocculation, sedimentation, creaming, ostwald ripening and coalescence among others. Researches on nanoemulsion wherein its particle size is reduced to nano-size have been actively conducted to resolve the instability of the emulsion. In terms of a preparation process, the known emulsions such as the Restasis™ are prepared by using a high speed agitator-type or a high speed shear-type apparatus such as a high pressure homogenizer or a microfluidizer which delivers great physical force to compositions while preparing the emulsion. As disclosed in the Korean Patent Publication No. 10-2008-0030828, this preparation process requires large manufacturing facility and heavy expenditure, and hardly applicable to heat-sensitive ingredients due to substantial temperature elevation caused by energies delivered to the emulsion while manufacturing. Also, cyclosporine emulsion prepared by this process was quickly flocculated due to unequal oil-drop size, and thus creaming process is accelerated and affects the long-term stability. In addition, there is a difficulty in securing uniform quality for each manufacturing lot because particle size distribution in a dispersed phase is relatively wide.

### PRIOR ART REFERENCE

### Patent Reference

U.S. Pat. No. 5,660,858
International Publication No. WO1995/31211
Korean Patent No. 10-1008189

### SUMMARY OF INVENTION

### Technical Problem

The inventors of the present disclosure confirmed that solubility of cyclosporine is increased, average particle size is formed in range of 1 nm to 100 nm and maximum particle size is formed in 220 nm or less, and the penetrance and efficacy can be improved and physicochemical stability, irritation, blurred vision and foreign body sensation can be effectively improved when ophthalmically administering the final product of nanoemulsion in case of preparing the ophthalmic nanoemulsion by appropriately mixing ingredients of the nanoemulsion while conducting a research on the ophthalmic nanoemulsion which can improve solubility of cyclosporine as an active ingredient, and accordingly completed the present disclosure.

Therefore, the present disclosure provides ophthalmic nanoemulsion compositions comprising: cyclosporine; one or more nonaqueoussolvents selected from a group consisting of vegetable oils, C₁₄-C₂₀ fatty acid esters and C₆-C₁₂ fatty acid esters of glycerol; one or more hydrophilic emulsifiers; one or more hydrophobic emulsifiers; and aqueous solvent.

The present disclosure also provides ophthalmic nanoemulsion compositions having average particle size range of 1 nm to 100 nm comprising: cyclosporine in the amount of 0.02 to 0.3 w/v % based on the total amount of the composition; one or more nonaqueous solvents in the amount of 0.1 to 2.5 w/v % based on the total amount of the composition selected from a group consisting of vegetable oils, C₁₄-C₂₀ fatty acid esters and C₆-C₁₂ fatty acid esters of glycerol; one or more hydrophilic emulsifiers in the amount of 0.1 to 5.0 w/v % based on the total amount of the composition selected from a group consisting of polyoxyethylene hydrogenated castor oils, polyoxyethylene sorbitan fatty acid esters and polyoxyethylene fatty acid esters; one or more hydrophobic emulsifiers in the amount of 0.1 to 5.0 w/v % based on the total amount of the composition selected from a group consisting of sorbitan fatty acid esters, glycerin fatty acid esters, diethylene glycol monoethyl ether, polyethylene glycols, propylene glycol and propylene glycol esters of fatty acids; and aqueous solvent.

The present disclosure also provides ophthalmic nanoemulsion compositions comprising: cyclosporine in the amount of 0.02 to 0.3 w/v % based on the total amount of the composition; castor oil in the amount of 8 times or more of said amount of cyclosporine to 2.5 w/v % or less of total amount of the composition; one or more hydrophilic emulsifiers selected from the group consisting of polyoxyethylene hydrogenated castor oils, polyoxyethylene sorbitan fatty acid esters and polyoxyethylene fatty acid esters; one or more hydrophobic emulsifiers selected from the group consisting of sorbitan fatty acid esters, glycerin fatty acid esters, diethylene glycol monoethyl ether, polyethylene glycols, propylene glycol and propylene glycol esters of fatty acids; and aqueous solvent.

The present disclosure also provides methods of preparing the ophthalmic nanoemulsion compositions having average particle size range of 1 nm to 100 nm comprising: preparation of a mixture compositions by stirring cyclosporine in the amount of 0.02 to 0.3 w/v % based on the total amount of the composition; one or more nonaqueoussolvents in the amount of 0.1 to 2.5 w/v % based on the total amount of the composition selected from the group consisting of the vegetable oils, C₁₄-C₂₀ fatty acid esters and C₆-C₁₂ fatty acid esters of glycerol; one or more hydrophilic emulsifiers in the amount of 0.1 to 5.0 w/v % based on the total amount of the composition selected from the group consisting of polyoxyethylene hydrogenated castor oils, polyoxyethylene sorbitan fatty acid esters and polyoxyethylene fatty acid esters; one or more hydrophobic emulsifiers selected from the group consisting of sorbitan fatty acid esters, glycerin fatty acid esters, diethylene glycol monoethyl ether, polyethylene glycols, propylene glycol and propylene glycol esters of fatty acids; and aqueous solvent.

The present disclosure also provides a method of preventing or treating ophthalmic diseases comprising an administration of the ophthalmic nanoemulsion compositions of the present disclosure to patients.

### Solution to Problem

The present disclosure provides ophthalmic nanoemulsion compositions comprising cyclosporine, the nonaqueous solvents, the emulsifiers and the aqueous solvent, more specifically the present disclosure provides the ophthalmic nanoemulsion compositions comprising: cyclosporine; one or more nonaqueous solvents selected from the group consisting of vegetable oils, C₁₄-C₂₀ fatty acid esters and C₆-C₁₂ fatty acid esters of glycerol; one or more hydrophilic emulsifiers; one or more hydrophobic emulsifiers; and the aqueous solvent.

The ophthalmic nanoemulsion compositions of the present disclosure can be prepared by appropriately mixing the above ingredients, and sterile filtration is available, solubility of cyclosporine is increased and stability is improved because its average particle (globule) size is formed in 200 nm or less or within the range of 1 nm to 100 nm and particle size distribution is narrow.

‘Cyclosporine’ is an active ingredient of the ophthalmic nanoemulsion compositions and can include cyclosporine A, cyclosporine A derivatives, cyclosporine B, cyclosporine C, cyclosporine D or its mixtures thereof among others, and preferably cyclosporine can be cyclosporine A or its derivatives thereof.

Cyclosporine can be contained in a therapeutically effective amount to improve dry eye syndrome and can be contained in 0.001 to 1.0 w/v %, 0.01 to 1.0 w/v % and preferably 0.02 to 0.3 w/v % based on the total ophthalmic nanoemulsion compositions for the purposes of the present disclosure.

With regard to preparation of an ophthalmic preparation, a composition wherein surfactant such as polyoxyethylated castor oils and polyoxyethylene sorbitan fatty acid esters among others is used for preventing precipitation of cyclosporine in the eyes after administration of a eye drop is disclosed, but its demerits are also known that the ophthalmic composition would exist in status of milky-colored opaque emulsion when using this composition and accordingly inducing blurred vision at initial stage of administration. Therefore, the ophthalmic nanoemulsion compositions are prepared through the present disclosure by properly selecting the nonaqueous solvent, the hydrophilic emulsifier and the hydrophobic emulsifier to resolve the above problems.

The ‘nonaqueous solvent’ can be at least one selected from the group consisting of vegetable oils, C₁₄-C₂₀ fatty acid esters and C₆-C₁₂ fatty acid ester glycerides among others. Specifically, the vegetable oils can be castor oil, coconut oil, cinnamon oil, corn oil, olive oil, cotton seed oil and soybean oil; the C₁₄-C₂₀ fatty acid esters can be lauric acid, myristic acid, palmitic acid, palmitoleic acid, stearic acid, oleic acid, linoleic acid, α-linolenic acid, γ-linolenic acid, eicosapentaenoic acid, ethyl oleate, isopropyl myristate; C₆-C₁₂ fatty acid esters of glycerol can be fatty acid ester glycerides such as labrafac PG and labrafac lipophile WL 1349 among others, caprylic acid-capric acid triglycerides such as miglyol 812, caprylic acid-capric acid-linoleic acid triglycerides such as miglyol 818. Also, preferably one or more nonaqueous solvents can be selected from a group consisting of castor oil, miglyol 812, ethyl oleate, isopropyl myristate, labrafac PG and labrafac lipophile WL 1349. Most preferably, the nonaqueous solvent is castor oil and it is commercially available under product name of castor oil (manufactured by ITHO oil chem., Japan). Castor oil decreases tear evaporation at the ocular surface and has superior spreadability compared to other oils, and thus castor oil is useful for treating dry eye syndrome such as a meibomian gland dysfunction at the lacrimal gland among others. However, the ophthalmic composition comprising the nonaqueous solvent such as castor oil among others might induce pains including an ophthalmic irritation, and visual disturbance. Therefore, it is desirable to use the nonaqueous solvent (i.e. castor oil among others) for the present disclosure in minimum concentration by which cyclosporine can be dissolve properly and adverse reactions can be minimized. By using the nonaqueous solvent in minimum concentration, the amount of the emulsifier can also be minimized which is used for stabilizing an oil phase, therefore it is available to provide the safer ophthalmic nanoemulsion compositions compared to cyclosporine emulsions on the market. The amount of the nonaqueous solvent can be 0.01-10.0 w/v %, 0.1-5.0 w/v %, and preferably 0.1-2.5 w/v % based on the total amount of the composition. Additionally, in case when the nonaqueous solvent is castor oil, the amount of castor oil can be 8 times or more of the amount of cyclosporine as active ingredient to 2.5 w/v % or less based on the total amount of the composition. The most stable nanoemulsion composition is formed when the amount of castor oil (i.e. the nonaqueous solvent) is 8 times or more of the amount of cyclosporine to 2.5 w/v % or less based on the total amount of the composition, therefore this amount is desirable to prepare the stable nanoemulsion composition of the present disclosure.

The nanoemulsion compositions of the present disclosure comprise one or more emulsifiers that help emulsification of the nonaqueous solvent in the aqueous solvent. One or more emulsifiers can be selected by considering ratio of Hydrophilic-Lipophilic Balance (hereinafter, HLB) values of each emulsifier depending on a required HLB value of the nonaqueous solvent, preferably depending on the required HLB value of castor oil. One or more emulsifiers can be selected from a group of the hydrophilic emulsifiers in which the HLB value is at least 8, specifically 10 or more, and from a group of the hydrophobic emulsifiers in which the HLB value is less than 8, specifically 6 or less.

The emulsifier of the present disclosure can be the hydrophilic emulsifier, the hydrophobic emulsifier or the mixtures thereof.

The hydrophilic emulsifier can be fatty acid, ester, ether, acid or its random combination thereof. For instance, polyoxyethylene hydrogenated castor oils, polyoxyethylene sorbitan fatty acid esters, polyoxyethylene fatty acid esters, fatty acid macrogol glycerides, caprylocaproyl polyoxylglyceride, poloxamers, tyloxapol and vitamin E TPGS (D-alpha tocopheryl polyethylene glycol 1000 succinate) among others can be used as the hydrophilic emulsifier but not limited to the above. Preferably, one or more hydrophilic emulsifiers can be selected from the group consisting of polyoxyethylene hydrogenated castor oil, polyoxyethylene sorbitan fatty acid ester and polyoxyethylene fatty acid ester, and also preferably, the hydrophilic emulsifier can be polyoxyl 35 hydrogenated castor oil or polyoxyethylene sorbitan monooleate, and both are being sold in the market under the product name of Cremophor EL™ or ELP™ (BASF), and Polysorbate 80 (NOF Corporation). The amount of the hydrophilic emulsifier can be 0.01-10.0 w/v %, 0.01-7.0 w/v % based on the total amount of the composition and most preferably 0.1-5.0 w/v %. In case when the nonaqueous solvent is castor oil, one or more hydrophilic emulsifiers can be selected from the group consisting of polyoxyethylene hydrogenated castor oil, polyoxyethylene sorbitan fatty acid ester and polyoxyethylene fatty acid ester, and when the hydrophilic emulsifier is polyoxyethylene hydrogenated castor oil, the amount of polyoxyethylene hydrogenated castor oil is desirable to be at least 1.6 times or more of the amount of castor oil (i.e. the nonaqueous solvent) to 5.0 w/v % or less based on the total amount of the composition. Also, when the nonaqueous solvent is castor oil and the hydrophilic emulsifier is polyoxyethylene hydrogenated castor oil, the amount of the hydrophilic emulsifier is desirable to be at least 12.8 times of the amount of cyclosporine (i.e. active ingredient) to 5.0 w/v % based on the total amount of the composition. A stable nanoemulsion having average particle size of 1 nm to 100 nm can be easily prepared with the contents stated above and show the best sensation by ocular instillation because the hydrophilic emulsifier is contained in 5.0 w/v % or less based on the total amount of the composition.

The hydrophobic emulsifier can be ionic or non-ionic, preferably non-ionic. Sorbitan fatty acid esters, diethylene glycol monoethyl ether, polyethylene glycols, propylene glycol, propylene glycol esters of fatty acids, glycerin fatty acid esters, oxy-alkanediols, lecithin and higher aliphatic alcohol (i.e. C₁₆ and greater) can be used as the hydrophobic emulsifier, and preferably one or more hydrophobic emulsifiers can be selected from the group consisting of sorbitan fatty acid esters, glycerin fatty acid esters, diethylene glycol monoethyl ether, polyethylene glycols, propylene glycols and propylene glycol esters of fatty acids. Specifically, the preferable hydrophobic emulsifiers are polyethylene glycols (PEG), propylene glycol, sorbitan fatty acid esters and diethylene glycol monoethyl ether, and these are being sold on the market as product names: Super Refined PEG 300™, Super Refined PEG 400™, Super Refined PEG 600™ (Croda), Propylene Glycol (Merck), Span 20, Span 80 (Croda) and Transcutol P (Gattefosse), respectively. The amount of said hydrophobic emulsifier can be 0.01-7.0 w/v % based on the total amount of the composition, and preferably it can be 0.1-5.0 w/v % based on the total amount of the composition. Additionally, the hydrophobic emulsifier can be polyethylene glycol, propylene glycol and diethylene glycol monoethyl ether, and the amount of these can be at least 0.1 w/v % or more based on the total amount of the composition, and it is preferable to be contained in 3 times or less than the amount of the hydrophilic emulsifier, which would preferably be polyoxyethylene hydrogenated castor oil. Also, in this case, it is most preferable that does not exceed 5.0 w/v % based on the total amount of the composition. It is most preferable that the hydrophobic emulsifier is contained at least 0.1 w/v % or more based on the total amount of the composition to prepare a stable nanoemulsion composition, and it is preferable that the hydrophobic emulsifier is contained 3 times or less than the amount of the hydrophilic emulsifier, preferably polyoxyethylene hydrogenated castor oil and 5.0 w/v % or less based on the total amount of the composition in order to achieve superior sensation by ocular instillation.

The aqueous solvent of the present disclosure is an adequate ingredient for preparation of ophthalmic preparations, and it can be sterile purified water, saline solution and water for injection.

Therefore, the present disclosure can provide the ophthalmic nanoemulsion composition having average particle size of 1 nm or more to 100 nm or less comprising: cyclosporine in the amount of 0.02-0.3 w/v % based on the total amount of the composition; one or more nonaqueous solvents in the amount of 0.1-2.5 w/v % based on the total amount of the composition that is selected from the group consisting of vegetable oils, C₁₄₋₂₀ fatty acid esters and C₆-C₁₂ fatty acid esters of glycerol; one or more hydrophilic emulsifiers in the amount of 0.1-5.0 w/v % based on the total amount of the composition that is selected from the group consisting of polyoxyethylene hydrogenated castor oils, polyoxyethylene sorbitan fatty acid esters and polyoxyethylene fatty acid esters; one or more hydrophobic emulsifiers in the amount of 0.1-5.0 w/v % based on the total amount of the composition that is selected from the group consisting of sorbitan fatty acid esters, glycerin fatty acid esters, diethylene glycol monoethyl ether, polyethylene glycols, propylene glycol and propylene glycol esters of fatty acids; and the aqueous solvent.

The nonaqueous solvent of the present disclosure can be at least one selected from the group consisting of castor oil, labrafac, miglyol 812, ethyl oleate and isopropyl myristate, and preferably the nonaqueous solvent is castor oil.

Additionally, the hydrophilic emulsifier can be preferably polyoxyethylene hydrogenated castor oils or polyoxyethylene sorbitan fatty acid esters, and the hydrophobic emulsifier can be at least one selected from the group consisting of polyethylene glycols, propylene glycol and diethylene glycol monoethyl ether.

In addition, the present disclosure can further comprise a stabilizer in the ophthalmic nanoemulsion composition. Physicochemical stabilities of the ophthalmic nanoemulsion composition of the present disclosure can be improved more with the additional inclusion of the stabilizer. The stabilizer can provide viscosity through gridding oil-drop of the nanoemulsion by forming a certain bonding structure via hydration in the aqueous solvent, and can act as physically stabilizing the nanoemulsion. The stabilizer can comprise cellulose-based compounds including carboxymethyl cellulose (CMC), hydroxypropyl methylcellulose (HPMC) and hydroxyethylcellulose (HEC) among others; polyvinyl-based compounds including polyvinyl alcohol (PVA) and polyvinylpyrrolidone (PVP) among others; acrylic-based compounds including carbomer among others; gum-based compounds including gellan gum and xanthan gum among others; polysaccharides including hyaluronic acid (HA), sodium hyaluronate, sodium alginate and dextran among others; or its random combinations thereof. Additionally, the stabilizer can be at least one selected from a group consisting of carboxymethyl cellulose, xanthan gum, hyaluronic acid (HA) and sodium hyaluronate.

Therefore, the composition of the present disclosure can comprise cyclosporine, the nonaqueous solvent, the hydrophilic emulsifier, the hydrophobic emulsifier, the stabilizer and the aqueous solvent.

Additionally, the composition of the present disclosure can be the ophthalmic nanoemulsion composition comprising: cyclosporine; one or more nonaqueoussolvents selected from the group consisting of vegetable oils, C₁₄-C₂₀ fatty acid esters and C₆-C₁₂ fatty acid esters of glycerol; one or more hydrophilic emulsifiers; one or more hydrophobic emulsifiers; the stabilizer selected from the group consisting of the cellulose-based compounds including carboxymethyl cellulose (CMC), hydroxypropyl methylcellulose (HPMC) and hydroxyethylcellulose (HEC) among others, the polyvinyl-based compounds including polyvinyl alcohol (PVA) and polyvinylpyrrolidone (PVP) among others; the acrylic-based compounds including carbomer among others; the gum-based compounds including gellan gum and xanthan gum among others; the polysaccharides including hyaluronic acid (HA), sodium hyaluronate, sodium alginate and dextran among others, and its mixtures; and the aqueous solvent.

The amount of the stabilizer can be 0.001-10.0 w/v %, 0.01-5.0 w/v %, and preferably 0.01-2.0 w/v % based on the total amount of the composition.

In addition, the ophthalmic nanoemulsion compositions of the present disclosure can further comprise pH modifiers, isotonizing agents, preservatives and buffering agents among others.

The pH adjusting agent can be sodium hydroxide and hydrochloric acid among others, and it can be used to obtain a proper pH value by adding a necessary amount via methods known in the art.

The isotonizing agent can be at least one selected from a group consisting of glycerol, mannitol, sorbitol, sodium chloride, potassium chloride, boric acid and borax, and the amount of the isotonizing agent can be in range of 0.01-10.0 w/v % based on the total amount of the composition, and can be used in 0.1-3.0 w/v %.

The preservative of the present disclosure can be quaternary ammonium compounds including benzalkonium chloride, benzethonium chloride, cetalkonium chloride and polyquaternium-1 (e.g. Polyquad®) among others; guanidine-based compounds including PHMB and chlorohexidine among others; chlorobutanol; mercury-based preservatives including thimerosal, phenyl mercury acetate and phenylmercuric nitrate among others; and oxidative preservatives including stabilized oxychloro complex (e.g. Purite®) and p-oxybenzoic acid alkyls (e.g. p-oxybenzoic acid methyl (PM)) among others.

The buffering agent of the present disclosure can be any buffering agents which are used for eye drop without any restrictions. There are an acetate buffer, a citrate buffer, a phosphate buffer (e.g. sodium phosphate or its hydrate, and sodium dihydrogen phosphate or its hydrate) and a borate buffer such as boric acid or its salt among others but not limited to these. The amount of the buffering agent can be adequately selected by those skilled in the art and it can be added in 0.001-10 w/v %, preferably 0.01-5.0 w/v %, and more preferably 0.1-2.0 w/v % based on the total amount of the composition.

In addition, it is preferable that the particle size of the ophthalmic nanoemulsion compositions of the present disclosure is 220 nm or less, and the particle size in the compositions can be 0 nm to 220 nm, and 0.3 nm to 220 nm.

Additionally, the present disclosure provides the ophthalmic nanoemulsion composition comprising: cyclosporine in the amount of 0.02-0.3 w/v % based on the total amount of the composition; castor oil that is contained in 8 times or more of the amount of said cyclosporine to 2.5 w/v % or less based on the total amount of the composition; one or more hydrophilic emulsifiers selected from the group consisting of polyoxyethylene hydrogenated castor oils, polyoxyethylene sorbitan fatty acid esters and polyoxyethylene fatty acid esters; one or more hydrophobic emulsifiers selected from a group consisting of sorbitan fatty acid esters, glycerin fatty acid esters, diethylene glycol monoethyl ether, polyethylene glycols, propylene glycol and propylene glycol esters of fatty acids; and the aqueous solvent.

The hydrophilic emulsifier can be polyoxyethylene hydrogenated castor oil and can be contained in 1.6 times or more of the amount of said castor oil to 5 w/v % or less based on the total amount of the composition.

Also, the hydrophobic emulsifier is at least one selected from the group consisting of polyethylene glycols, propylene glycol and diethylene glycol monoethyl ether, and can be contained in 0.1 w/v % of total amount of the composition to 3 times or less of the amount of the hydrophilic emulsifier.

In addition, the present disclosure provides the ophthalmic nanoemulsion composition: wherein the hydrophilic emulsifier is the polyoxyethylene hydrogenated castor oil and contained in 12.8 times or more of the amount of said cyclosporine to 5 w/v % or less based on the total amount of the composition; and the hydrophobic emulsifier is at least one selected from the group consisting of polyethylene glycols, propylene glycol and diethylene glycol monoethyl ether and contained in 0.1 w/v %-5 w/v % based on the total amount of the composition.

Additionally, the present disclosure provides a method of preventing or treating ophthalmic diseases comprising administration into eye of the ophthalmic nanoemulsion composition to patients.

The ophthalmic diseases can be intrinsic diseases or extrinsic diseases due to external injury or wearing of hard contact lens, and preferably the ophthalmic disease can be Sjogren syndrome or dry eye syndrome, and more preferably, the ophthalmic disease can be dry eye syndrome.

Additionally, the present disclosure provides a method of preparation of the ophthalmic nanoemulsion composition having average particle size of 1 nm-100 nm comprising: preparing a mixture by stirring cyclosporine, the nonaqueous solvent, the hydrophilic emulsifier, the hydrophobic emulsifier and the aqueous solvent.

Specifically, the present disclosure provides a method of preparation of the ophthalmic nanoemulsion composition having average particle size of 1 nm-100 nm comprising: preparing the mixture by dissolving cyclosporine (i.e. active ingredient) in the nonaqueous solvent, adding the hydrophilic emulsifier, the hydrophobic emulsifier and the aqueous solvent to the dissolved composition, and stirring the same.

More specifically, the present disclosure provides a method of preparation of the ophthalmic nanoemulsion composition having average particle size of 1 nm-100 nm comprising: preparing the mixture by stirring cyclosporine in the amount of 0.02-0.3 w/v % based on the total amount of the composition; one or more nonaqueoussolvents in the amount of 0.1-2.5 w/v % based on the total amount of the composition selected from the group consisting of vegetable oils, C₁₄-C₂₀ fatty acid esters and C₆-C₁₂ fatty acid esters of glycerol; one or more hydrophilic emulsifiers in the amount of 0.1 to 5.0 w/v % based on the total amount of the composition selected from the group consisting of polyoxyethylene hydrogenated castor oils, polyoxyethylene sorbitan fatty acid esters and polyoxyethylene fatty acid esters; one or more hydrophobic emulsifiers selected from the group consisting of sorbitan fatty acid esters, glycerin fatty acid esters, diethylene glycol monoethyl ether, polyethylene glycols, propylene glycol and propylene glycol esters of fatty acids; and the aqueous solvent.

In the above preparation of said mixture, the method of preparation can further comprise additional dissolving of the stabilizer or the isotonizing agent in the aqueous solvent, stirring of the same with the prepared mixture and controlling of its pH scale.

According to the preparation method of the present disclosure, the nanoemulsion having average particle size of 1 nm-100 nm can be formed because ingredients are adequately mixed; a common sterilizing filtration by using a 0.22 μm filter is available without using an existing high speed stirrer-type or a high speed shear-type apparatus such as a high pressure homogenizer or a microfluidizer because the ophthalmic nanoemulsion composition having a maximum particle size of 220 nm or less is prepared; and the cost of preparing said ophthalmic nanoemulsion composition having a particle size of 220 nm or less is low.

The nanoemulsion composition prepared through the present disclosure has superior effects such as little irritation, foreign body sensation and blurred vision among others, and releases cyclosporine A (i.e. active ingredient) with an appropriate rate at the cellulose membrane-release assay which evaluates release of drugs.

The nanoemulsion composition prepared through the present disclosure can be effectively used for treating dry eye syndrome because patients' inappropriate irritant and foreign body sensation are improved as well as exhibiting high treatment effect in treating dry eye syndrome when using said nanoemulsion composition as an ophthalmic composition for ocular administration, and the nanoemulsion composition would increase tear secretion and retention time of tear film. Also, the amount of the cyclosporine A residues in ocular tissue are expected to be high after administration of the nanoemulsion composition.

### Advantageous Effect

The ophthalmic nanoemulsion composition of the present disclosure is having the average particle size of 200 nm or less, preferably 100 nm or less, and the particle distribution is characterized in narrow. Therefore, the ophthalmic nanoemulsion composition of the present disclosure can be effectively used for ophthalmic composition because sterilizing filtration is available, stability is improved and the effect of improving foreign body sensation and blurred vision is clinically superior.

### BRIEF DESCRIPTION OF DRAWINGS

FIG. 1 illustrates a confirmed particle size distribution of the ophthalmic nanoemulsion composition of Example 33 and Restasis™ eye drop.

FIG. 2 illustrates a confirmed distribution stability of the ophthalmic nanoemulsion compositions of Examples 62 and 63, the emulsion and the suspension through Turbiscan Stability Index (TSI).

FIG. 3 is a graph showing measured average values of burning sensation and foreign body sensation of Example 62 and the Restasis™ which is a control drug through testing sensation of drop into the eyes.

### DESCRIPTION OF EMBODIMENTS

The present disclosure will be described more fully hereinafter with reference to the accompanying examples. However, the following examples are intended to illustrate the present invention, and the present invention is not limited by the following examples.

### Experimental Example 1

### Preparation of the Nanoemulsion and Measurement of an Average Particle Size of the Same in Accordance with Type and Content Variation of the Nonaqueous Solvent

The nanoemulsion compositions were prepared with different amount of castor oil, labrafac lipophils WL 1349 or miglyol 812, and then measured its average particle size. Specific method of preparing the nanoemulsion composition follows. Cyclosporine A and the nonaqueous solvent were mixed in the amount stated in Table 1 below and completely dissolved under 600-800 rpm and 70° C. by using a stirrer (Super-Nuova™ Multi-place, Thermo Scientific). An oil phase was prepared by adding the hydrophilic and hydrophobic emulsifiers with the contents stated in Table 1 to the above prepared mixed solution and sufficiently mixing via stirring the same. The prepared solution was cooled at room temperature and the oily phase was put into the aqueous solvent for washing the oily phase several times, and then stirred it under 400-500 rpm and room temperature by using the stirrer (Super-Nuova™ Multi-place, Thermo Scientific). The aqueous solvent was added until the final volume was to be 100 mL after 30 minutes or more of stirring. The nanoemulsion voluntarily formed a stable homogeneous phase through Self Nano-Emulsifying Drug Delivery system (SNEDDS). The size of the particle of the ophthalmic nanoemulsion prepared through the above method was measured by using the Zetasizer (Malvern Instruments, England), which is an apparatus for measuring particle sizes, and the constitution and measured average particle size (nm) of the prepared nanoemulsion composition are shown in Table 1 below.

TABLE 1

| | | Example | | | | | | | | | | | | | | |
|---|---|---|---|---|---|---|---|---|---|---|---|---|---|---|---|---|
| Ingredient | | 1 | 2 | 3 | 4 | 5 | 6 | 7 | 8 | 9 | 10 | 11 | 12 | 13 | 14 | 15 |
| Active | Cyclosporine | 0.05 | 0.03 | 0.1 | 0.05 | 0.05 | 0.05 | 0.05 | 0.05 | 0.03 | 0.05 | 0.05 | 0.05 | 0.05 | 0.05 | 0.03 |
| Ingredient | | | | | | | | | | | | | | | | |
| (w/v %) | | | | | | | | | | | | | | | | |
| Nonaqueous | Castor Oil | 0.42 | 0.25 | 0.84 | | | | | | | | | | | | |
| Solvent | Labrafac | | | | 2 | 1.8 | 1.8 | 1.8 | 2 | 1.2 | | | | | | |
| (w/v %) | Miglyol 812 | | | | | | | | | | 0.75 | 0.66 | 0.66 | 0.66 | 0.75 | 0.45 |
| Hydrophilic | Polysorbate80 | | | | | | | | 1.6 | 0.8 | | | | | 1.6 | 0.8 |
| Emulsifier | Cremophor | 1 | 1 | 4.8 | 2.4 | 2.4 | 1.6 | 1.6 | | | 2.4 | 2.4 | 1.6 | 1.6 | | |
| (w/v %) | ELP | | | | | | | | | | | | | | | |
| Hydrophobic | Polyethylene | 0.4 | 0.3 | 0.8 | 0.6 | 0.4 | 0.4 | | 0.4 | 0.2 | 0.6 | 0.4 | 0.4 | | 0.4 | 0.2 |
| Emulsifier | Glycol 400 | | | | | | | | | | | | | | | |
| (w/v %) | Propylene | 0.3 | 0.2 | 0.6 | | 0.3 | 0.3 | | | | | 0.3 | 0.3 | | | |
| | Glycol | | | | | | | | | | | | | | | |
| | Transcutol P | | | | | | | 0.4 | | | | | | 0.4 | | |
| Aqueous | Purified | q.s | q.s | q.s | q.s | q.s | q.s | q.s | q.s | q.s | q.s | q.s | q.s | q.s | q.s | q.s |
| Solvent | Water | | | | | | | | | | | | | | | |
| | Average | 41.05 | 31.04 | 35.29 | 45.92 | 48.33 | 58.51 | 54.21 | 76.15 | 73.25 | 30.72 | 32.76 | 42.16 | 44.8 | 62.95 | 60.31 |
| | Particle Size | | | | | | | | | | | | | | | |

All of the particle sizes of the nanoemulsion compositions in Examples 1 to 15 were shown as 220 nm or less in maximum, and as shown in above Table 1, it was confirmed that the average particle size were all 100 nm or less in particular and transparent nanoemulsion compositions were prepared. The nanoemulsion including cyclosporine as a active ingredient can be determined to have a suitable particle size when the maximum particle size is 220 nm or less, and thus it was confirmed that the ophthalmic nanoemulsion composition having a very small average particle size can be prepared according to the compositions of the present disclosure. In particular, Examples 1 to 3 exhibited superior effect in forming the nanoemulsion where the average particle sizes were 41.05, 31.04 and 35.29 nm while the examples were consisted as including castor oil having relatively low amount of 0.42, 0.25 and 0.84 w/v % compared to the other nonaqueous solvents (i.e. labrafac and miglyol 812) and accordingly consisted of the emulsifier having low content. When considering that the nonaqueous solvent can give irritation upon administration into eye, castor oil has an advantage that it can minimize the irritation caused by an oil component in ophthalmic compositions, and thus it was confirmed that castor oil is one of the preferred nonaqueous solvent for the ophthalmic nanoemulsion composition.

### Experimental Example 2

### Preparation of the Nanoemulsion and Measurement of an Average Particle Size of the Same According to Type and Varying Amount of the Emulsifier

The nanoemulsion composition of Examples 16 to 36 were Prepared by the same method explained in Experimental Example 1 via using castor oil with the nonaqueous solvent, and varying amount of castor oil and type and amount of the emulsifier. Constitution and amount of the prepared nanoemulsion composition are shown in Table 2 below.

TABLE 2

| | | Example | | | | | | | | | | |
|---|---|---|---|---|---|---|---|---|---|---|---|---|
| Ingredient | | 16 | 17 | 18 | 19 | 20 | 21 | 22 | 23 | 24 | 25 | 26 |
| Active | Cyclosporine | 0.05 | 0.05 | 0.05 | 0.05 | 0.05 | 0.03 | 0.1 | 0.05 | 0.05 | 0.05 | 0.05 |
| ingredient | | | | | | | | | | | | |
| (w/v %) | | | | | | | | | | | | |
| Nonaqueous | Castor Oil | 0.42 | 0.42 | 0.42 | 0.42 | 0.42 | 0.25 | 0.84 | 0.42 | 0.42 | 0.42 | 0.42 |
| Solvent | | | | | | | | | | | | |
| (w/v %) | | | | | | | | | | | | |
| Hydrophilic | Polysorbate | 1.6 | 0.4 | 1.4 | 0.8 | 0.8 | 0.4 | 1.6 | | | | |
| Esulsifier | 80 | | | | | | | | | | | |
| (w/v %) | Cremophor | | | | | | | | 2.4 | 1.6 | 1.2 | 1.6 |
| | ELP | | | | | | | | | | | |
| Hydrophobic | Span 80 | 0.4 | 0.1 | | | | 0.1 | 0.4 | 0.6 | 0.4 | 0.8 | |
| Emulsifier | Span 20 | | | 0.6 | | | | | | | | |
| (w/v %) | Polyethylene | | | | | | | | | | | |
| | Glycol 300 | | | | | | | | | | | 0.4 |
| | Polyethylene | | | | 0.2 | | | | | | | |
| | Glycol 400 | | | | | | | | | | | |
| | Propylene | | | | | | | | | | | |
| | Glycol | | | | | | | | | | | |
| | Transcutol P | | | | | 0.2 | | | | | | |
| Aqueous | Purified | q.s | q.s | q.s | q.s | q.s | q.s | q.s | q.s | q.s | q.s | q.s |
| Solvent | Water | | | | | | | | | | | |
| | Average | 73.52 | 84.3 | 59.18 | 67.64 | 67.21 | 61.94 | 73.46 | 30.11 | 43.41 | 48.5 | 49.26 |
| | Particle | | | | | | | | | | | |
| | Size | | | | | | | | | | | |

| | | Example | | | | | | | | | |
|---|---|---|---|---|---|---|---|---|---|---|---|
| Ingredient | | 27 | 28 | 29 | 30 | 31 | 32 | 33 | 34 | 35 | 36 |
| Active | Cyclosporine | 0.05 | 0.05 | 0.05 | 0.05 | 0.03 | 0.1 | 0.05 | 0.05 | 0.05 | 0.05 |
| ingredient | | | | | | | | | | | |
| (w/v %) | | | | | | | | | | | |
| Nonaqueous | Castor Oil | 0.42 | 0.42 | 0.42 | 0.42 | 0.25 | 0.84 | 0.42 | 0.42 | 0.42 | 0.42 |
| Solvent | | | | | | | | | | | |
| (w/v %) | | | | | | | | | | | |
| Hydrophilic | Polysorbate | | | | | | | | | 0.8 | |
| Esulsifier | 80 | | | | | | | | | | |
| (w/v %) | Cremophor | 2.4 | 1.6 | 2.4 | 1.6 | 1.2 | 4.8 | 2.4 | 1.8 | | 2.4 |
| | ELP | | | | | | | | | | |
| Hydrophobic | Span 80 | | | | | | | | | | |
| Emulsifier | Span 20 | | | | | | | | | | |
| (w/v %) | Polyethylene | | | | | | | | | | |
| | Glycol 300 | 0.6 | | | | | | | | | |
| | Polyethylene | | 0.4 | 0.6 | | 0.3 | 1.2 | 0.4 | 0.4 | | |
| | Glycol 400 | | | | | | | | | | |
| | Propylene | | | | | | | 0.3 | 0.3 | | |
| | Glycol | | | | | | | | | | |
| | Transcutol P | | | | 0.4 | | | | | | |
| Aqueous | Purified | q.s | q.s | q.s | q.s | q.s | q.s | q.s | q.s | q.s | q.s |
| Solvent | Water | | | | | | | | | | |
| | Average | 40.38 | 41.5 | 19.53 | 25.04 | 39.62 | 25.5 | 31.6 | 32.25 | 118.3 | 98.52 |
| | Particle | | | | | | | | | | |
| | Size | | | | | | | | | | |

As shown in Table 2, it was confirmed that the average particle size of the nanoemulsion composition in Examples 16 to 34 were 100 nm or less, and that its particle size distribution was very narrow. Therefore, it was confirmed that the ophthalmic nanoemulsion composition having average particle size of 100 nm or less and narrow particle distribution can be prepared when mixing cyclosporine, castor oil, the hydrophilic emulsifier, the hydrophobic emulsifier and the aqueous solvent with the above constitution.

In Table 2, Examples 35 and 36 are the compositions prepared by only using the hydrophilic emulsifier except the hydrophobic emulsifier and accordingly an opaque emulsion having wide particle size distribution was formed, and especially, it was confirmed that Example 36 had very unsuitable particle distribution because its particle size distribution was wide as 17.02-382.5 nm. Therefore, it was confirmed that combination of the hydrophilic emulsifier and the hydrophobic emulsifier is preferred to prepare the nanoemulsion having a suitable particle size and particle distribution.

It is important to have a suitable particle size distribution to prepare the ophthalmic composition, and thus the particle size distribution of Example 33 which was prepared by the method of the present disclosure was compared to that of the commercially available Restasis™ eye drop.

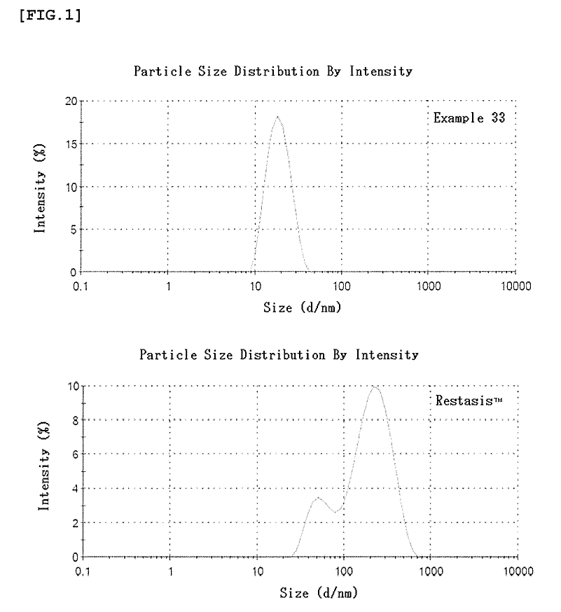

The result of the comparison is illustrated in FIG. 1.

As illustrated in FIG. 1, the composition according to the present disclosure (Example 33) had very narrow particle size distribution of 8.721-43.82 nm while that of the Restasis™ eye drop was very wide as 21.04-712.4 nm.

Namely, both that the nanoemulsion composition of the present disclosure can form more suitable composition for preparation of ophthalmic composition compared to the Restasis™ eye drop and that the sterilizing filtration by using 0.22 μm filter was available due to the maximum particle size of the nanoemulsion composition of the present disclosure as 220 nm or less, were confirmed.

### Experimental Example 3

### Optimum Content of the Ingredients of the Nanoemulsion Composition

Formation of the nanoemulsion depending on variation of amount of each ingredient was confirmed to determine relative amount of the nonaqueous solvent, the hydrophilic emulsifier and the hydrophobic emulsifier which are suitable for preparation of cyclosporine ophthalmic nanoemulsion composition of the present disclosure.

3.1 Content of the Nonaqueous Solvent

Composition were prepared by using the same method explained in Experimental Example 1 with fixed contents of cyclosporine, the hydrophilic emulsifier and the hydrophobic emulsifier and varied amount of castor oil to 2.5, 3.0 and 3.5 w/v %, and formation of the nanoemulsion was confirmed.

The result is shown in Table 3 below.

TABLE 3

| | | | |
|---|---|---|---|
| Cyclosporine | 0.05 g | 0.05 g | 0.05 g |
| Castor Oil | 2.5 g | 3.0 g | 3.5 g |
| Cremophor ELP | 5.0 g | 5.0 g | 5.0 g |
| PEG 400 | 2.0 g | 2.0 g | 2.0 g |
| Water for | q.s −> | q.s −> | q.s −> |
| Injection | 100 ml | 100 ml | 100 ml |
| Average Particle | 45.75 | 105.0 | N/A |
| Size (nm) | | | |

As shown in Table 3, formation of the superior nanoemulsion having a particle size of 45.75 nm was confirmed when comprising 2.5 w/v % of castor oil based on the total amount of the composition, but the particle size was increased when increasing the amount of castor oil to 3.0 w/v % and the particle size could not be measured when increasing the amount of castor oil up to 3.5 w/v %. That is, it was confirmed that castor oil (i.e. the nonaqueous solvent) was preferred to be contained 8 times or more of cyclosporine (i.e. active ingredient) to prepare the nanoemulsion composition of the present disclosure, and the maximum amount of castor oil in a composition including hydrophilic surfactant up to 5 w/v % was preferred to be 2.5 w/v % or less.

3.2 Amount of the Emulsifier

The hydrophilic emulsifier and the hydrophobic emulsifier are necessary ingredients to form the nanoemulsion but these incur decline of sensation by ocular instillation when contained in excessively, and the nanoemulsion cannot be formed when insufficiently contained, and thus it is important to select an adequate amount of the hydrophilic emulsifier and the hydrophobic emulsifier. Therefore, the amount of the hydrophilic emulsifier and the hydrophobic emulsifier which are necessary for preparing the ophthalmic nanoemulsion composition of the present disclosure were confirmed through a comparative experiment. The amount of castor oil (i.e. the nonaqueous solvent) was set to be 8 times or more of the amount of cyclosporine, and preparation was conducted under same conditions to Experimental Example 1 with constituting the Cremophor ELP as the hydrophilic emulsifier and PEG 400 as the hydrophobic emulsifier. Each content and result of the nanoemulsion formation therefrom is shown in Table 4 below.

TABLE 4

| Ingredient | 1 | 2 | 3 | 4 | 5 |
|---|---|---|---|---|---|
| Cyclosporine | 0.05 g | 0.05 g | 0.05 g | 0.05 g | 0.03 g |
| Castor Oil | 0.42 g | 0.42 g | 0.42 g | 0.42 g | 0.25 g |
| Cremophor ELP | 1.8 g | 1.8 g | 0.6 g | 0.4 g | 0.4 g |
| Polyethylene | 2.0 g | 5.0 g | 2.0 g | 2.5 g | 0.1 g |
| glycol 400 | | | | | |
| Water for | q.s −> | q.s −> | q.s −> | q.s −> | q.s −> |
| Injection | 100 ml | 100 ml | 100 ml | 100 ml | 100 ml |
| Average Particle | 22.96 | 27.25 | 74.53 | 127.9 | 48.67 |
| Size (nm) | | | | | |
| Note | — | — | Wide Particle | — | — |
| | | | Distribution | | |
| | | | Formation | | |
| | | | of Opaque | | |
| | | | Composition | | |

As shown in Table 4, the nanoemulsion having an average particle size of 100 nm or less was formed in every composition including Cremophor ELP (i.e. the hydrophilic emulsifier) 1.6 times or more of the amount of castor oil or including the PEG 400 (i.e. the hydrophobic emulsifier) in maximum of 3 times or less of the amount of the hydrophilic emulsifier, but confirmed that formation of the nanoemulsion in composition wherein the amount of Cremophor ELP is less than 1.6 times of the amount of castor oil and the amount of PEG 400 (i.e. the hydrophobic emulsifier) exceeded 3 times of the amount of Cremophor ELP (i.e. the hydrophilic emulsifier) was relatively difficult. Furthermore, the nanoemulsion was formed but could not maintain its phase due to low stability when contained the hydrophobic emulsifier less than 0.1 w/v % based on the total amount of the composition. Unpleasant feeling by ocular instillation gets worse when each amount of the hydrophilic emulsifier and the hydrophobic emulsifier was exceeded 5 w/v % based on the total amount of the composition, and thus it was confirmed that it is most preferable to include the hydrophilic emulsifier 1.6 times in minimum of the amount of castor oil (i.e. nonaqueous solvent) to 5 w/v % or less based on the total amount of the composition, and to include the hydrophobic emulsifier 0.1 w/v % based on the total amount of the composition to 3 times or less of the amount of the hydrophilic emulsifier and 5 w/v % or less based on the total amount of the composition to prepare the nanoemulsion composition having superior sensation by ocular instillation and stability.

### Experimental Example 4

### Stabilizer-Added Nanoemulsion Composition

As confirmed in Experimental Examples 1 through 3, the nanoemulsion composition having an average particle size of 100 nm or less can be prepared only by adding cyclosporine, the nonaqueous solvent, the hydrophilic emulsifier and the hydrophobic emulsifier. Furthermore, the stabilizer can be selectively added to prepare the ophthalmic nanoemulsion composition to improve stability because stable maintenance of particle size after preparation of the ophthalmic nanoemulsion composition is important. Therefore, it was confirmed whether the particle size of the nanoemulsion can be maintained when the stabilizer is added, and stability thereof.

4.1 Preparation of the Stabilizer-Added Nanoemulsion Composition and its Average Particle Size

Specifically, the nanoemulsion compositions were prepared according to the contents stated in Table 5 below. The stabilizer and the isotonizing agent were hydrated in the aqueous solvent and its pH was adjusted to 7.2 by using NaOH and HCl. The oily phase was prepared by using the same method to Experimental Example 1 wherein cyclosporine, the nonaqueous solvent and the emulsifier of the contents stated in Table 5 were completely dissolved, it was put into the aqueous solvent, and it was stirred by the stirrer (Super-Nuova™ Multi-place, Thermo Scientific) under conditions of 400-500 rpm and room temperature. Single phase was formed through Self Nano-Emulsifying Drug Delivery system (SNEDDS) as Experimental Example 1. Particle size of the prepared nanoemulsion composition was measured in the same manner to Experimental Example 1. Each average particle size of the nanoemulsion was shown in Table 5 below.

TABLE 5

| | | Example | | | | | | | |
|---|---|---|---|---|---|---|---|---|---|
| Ingredient | | 37 | 38 | 39 | 40 | 41 | 42 | 43 | 44 |
| Active | Cyclosporine | 0.05 | 0.05 | 0.05 | 0.05 | 0.05 | 0.05 | 0.05 | 0.05 |
| ingredient | | | | | | | | | |
| (w/v %) | | | | | | | | | |
| Nonaqueous | Castor Oil | 0.42 | 0.42 | 0.42 | 0.42 | 0.42 | 0.42 | 0.42 | 0.42 |
| Solvent | | | | | | | | | |
| (w/v %) | | | | | | | | | |
| Emulsifier | Cremophor ELP | 2.4 | 2.4 | 2.4 | 2.4 | 2.4 | 2.4 | 2.4 | 2.4 |
| (w/v %) | Polyethylene glycol 400 | 0.6 | 0.6 | 0.6 | 0.6 | 0.6 | 0.6 | 0.6 | 0.6 |
| Stabilizer | Sodium Hyaluronate | 0.1 | — | — | — | — | — | — | — |
| (w/v %) | Sodium Alginate | — | 0.2 | — | — | — | — | — | — |
| | Hydroxypropylmethylcellulose | — | — | 0.5 | — | — | — | — | — |
| | Polyvinylpyrrolidone | — | — | — | 1.2 | — | — | — | — |
| | Hydroxyethylcellulose | — | — | — | — | 0.5 | — | — | — |
| | Polyvinyl alcohol | — | — | — | — | — | 1.4 | — | — |
| | Xanthan Gum | — | — | — | — | — | — | 0.2 | — |
| | Sodium Carboxymethyl | — | — | — | — | — | — | — | 0.5 |
| | Cellulose | | | | | | | | |
| Isotonizing | Glycerol | 2.2 | 2.2 | 2.2 | 2.2 | 2.2 | 2.2 | 2.2 | 2.2 |
| Agent | | | | | | | | | |
| (v/v %) | | | | | | | | | |
| pH | NaOH, HCl | q.s | q.s | q.s | q.s | q.s | q.s | q.s | q.s |
| adjusting | | →7.2 | →7.2 | →7.2 | →7.2 | →7.2 | →7.2 | →7.2 | →7.2 |
| agent | | | | | | | | | |
| Aqueous | Purified Water(ml) | q.s | q.s | q.s | q.s | q.s | q.s | q.s | q.s |
| Solvent | | | | | | | | | |
| | Average Particle Size | 28.15 | 25.62 | 61.75 | 40.41 | 60.33 | 41.03 | 33.93 | 33.15 |

As shown in Table 5, the nanoemulsion composition having an average size of 100 nm or less was formed even with the addition of the stabilizer and it can be confirmed that its particle size distribution of the prepared composition was narrow.

Accordingly, it was confirmed that the preferred particle size and particle size distribution of the present disclosure can be maintained even if additionally comprising the stabilizer.

4.2 Evaluation of Thermal Stability of the Nanoemulsion Composition

The nanoemulsion compositions were prepared with the same content stated in Table 6 below by using the same method to Experimental Example 4(1). To evaluate physicochemical stabilities of the nanoemulsion composition, the amount of cyclosporine and average particle size of the nanoemulsion composition were analyzed while storing the nanoemulsion composition for 2 weeks under high temperature of 70±2° C. The amount of cyclosporine was measured by chromatography, the ACQUITY Ultra Pressure Liquid Chromatography (UPLC) system (Waters Asia Ltd., 396 Alexandra Road #04-06 BP Tower, Singapore 119954), under analysis condition stated in Table 7 below.

TABLE 6

| | | Example | | | | | | | | |
|---|---|---|---|---|---|---|---|---|---|---|
| Ingredient | | 45 | 46 | 47 | 48 | 49 | 50 | 51 | 52 | 53 |
| Active | Cyclosporine | 0.05 | 0.05 | 0.05 | 0.05 | 0.05 | 0.05 | 0.05 | 0.05 | 0.05 |
| ingredient | | | | | | | | | | |
| (w/v %) | | | | | | | | | | |
| Nonaqueous | Castor Oil | 0.42 | 0.42 | 0.42 | 0.42 | 0.42 | 0.42 | 0.42 | 0.42 | 0.42 |
| Solvent | | | | | | | | | | |
| (w/v %) | | | | | | | | | | |
| Emulsifier | Cremophor ELP | 2.4 | 2.4 | 2.4 | 2.4 | 2.4 | 2.4 | 2.4 | 2.4 | 2.4 |
| (w/v %) | Polyethylene | 0.6 | 0.6 | 0.6 | 0.6 | 0.6 | 0.6 | 0.4 | 0.4 | 0.4 |
| | glycol 400 | | | | | | | | | |
| | Propylene Glycol | | | | | | | 0.3 | 0.3 | 0.3 |
| Stabilizer | Sodium Hyaluronate | | 0.1 | | | | | | 0.1 | |
| (w/v %) | Polyvinylpyrrolidone | | | 1.8 | | | | | | 1.8 |
| | Sodium Alginate | | | | 0.2 | | | | | |
| | Sodium Carboxymethyl | | | | | 0.5 | | | | |
| | Cellulose | | | | | | | | | |
| | Polyvinylalcohol | | | | | | 1.4 | | | |
| | Xanthan Gum | | | | | | | | | |
| Isotonizing | Glycerol | 2 | 2 | 2 | 2 | 2 | 2 | 1.7 | 1.7 | 1.7 |
| Agent | | | | | | | | | | |
| (v/v %) | | | | | | | | | | |
| pH | NaOH, HCl | q.s | q.s | q.s | q.s | q.s | q.s | q.s | q.s | q.s |
| adjusting | | →7.2 | →7.2 | →7.2 | →7.2 | →7.2 | →7.2 | →7.2 | →7.2 | →7.2 |
| agent | | | | | | | | | | |
| Aqueous | Purified Water (ml) | q.s | q.s | q.s | q.s | q.s | q.s | q.s | q.s | q.s |
| Solvent | | | | | | | | | | |

| | | Example | | | | | | | |
|---|---|---|---|---|---|---|---|---|---|
| Ingredient | | 54 | 55 | 56 | 57 | 58 | 59 | 60 | 61 |
| Active | Cyclosporine | 0.05 | 0.05 | 0.05 | 0.05 | 0.05 | 0.05 | 0.05 | 0.05 |
| ingredient | | | | | | | | | |
| (w/v %) | | | | | | | | | |
| Nonaqueous | Castor Oil | 0.42 | 0.42 | 0.42 | 0.42 | 0.42 | 0.42 | 0.42 | 0.42 |
| Solvent | | | | | | | | | |
| (w/v %) | | | | | | | | | |
| Emulsifier | Cremophor ELP | 2.4 | 2.4 | 2.4 | 1.8 | 1.8 | 1.8 | 1.8 | 1.8 |
| (w/v %) | Polyethylene | 0.4 | 0.4 | 0.4 | 0.4 | 0.4 | 0.4 | 0.4 | 0.4 |
| | glycol 400 | | | | | | | | |
| | Propylene Glycol | 0.3 | 0.3 | 0.3 | 0.3 | 0.3 | 0.3 | 0.3 | 0.3 |
| Stabilizer | Sodium Hyaluronate | | | | | | | | |
| (w/v %) | Polyvinylpyrrolidone | | | | | 1.8 | | | |
| | Sodium Alginate | 0.2 | | | | | | | |
| | Sodium Carboxymethyl | | 0.5 | | | | 0.5 | 0.2 | |
| | Cellulose | | | | | | | | |
| | Polyvinylalcohol | | | 1.4 | | | | | |
| | Xanthan Gum | | | | | 0.1 | 0.1 | | 0.2 |
| Isotonizing | Glycerol | 1.7 | 1.7 | 1.7 | 1.7 | 1.7 | 1.7 | 1.7 | 1.7 |
| Agent | | | | | | | | | |
| (v/v %) | | | | | | | | | |
| pH | NaOH, HCl | q.s | q.s | q.s | q.s | q.s | q.s | q.s | q.s |
| adjusting | | →7.2 | →7.2 | →7.2 | →7.2 | →7.2 | →7.2 | →7.2 | →7.2 |
| agent | | | | | | | | | |
| Aqueous | Purified Water (ml) | q.s | q.s | q.s | q.s | q.s | q.s | q.s | q.s |
| Solvent | | | | | | | | | |

TABLE 7
Analytical Conditions of the UPLC

| Column | UPLC column 1HSS T3.8 μm 2.1 × 100 mm | |
|---|---|---|
| Wavelength of UV detector | 210 | nm |
| Column Temperature | 75° | C. |
| Sample Temperature | 25° | C. |
| Flow Rate | 0.250 | ml/min |
| Injection Volume | 5 | μl |
| Run Time | 20 | min |

| | |
|---|---|
| Mobile Phase | Acetonitrile(ACN):water = 68:32 |

Measurement results of content (%) of cyclosporine and average particle size (nm) in the nanoemulsion composition, which were stored for 2 weeks under high temperature, are shown in Table 8 below.

TABLE 8

| | Duration | Example | | | | | | | | |
|---|---|---|---|---|---|---|---|---|---|---|
| Item | (week) | 45 | 46 | 47 | 48 | 49 | 50 | 51 | 52 | 53 |
| Content | 0 | 101.97 | 102 | 101.66 | 98.32 | 99.91 | 98.32 | 101.87 | 100.6 | 100.36 |
| | 1 | 97.17 | 103.8 | 103.55 | 100.7 | 103.3 | 101.1 | 97.85 | 98.02 | 97.08 |
| | 2 | 92.96 | 99.93 | 102.47 | 96.83 | 94.64 | 96.83 | 90.32 | 91.86 | 98.73 |
| Size | 0 | 17.85 | 30.59 | 25.41 | 24.06 | 33.15 | 36.11 | 18.53 | 32.64 | 30.14 |
| | 1 | 30.12 | 27.9 | 28.26 | 24.34 | 35.15 | 50.85 | 21.02 | 34.57 | 31.66 |
| | 2 | 50.04 | 23.92 | 29.86 | 23.63 | 33.66 | 50.8 | 53.06 | 34.29 | 38.84 |

| | Duration | Example | | | | | | | |
|---|---|---|---|---|---|---|---|---|---|
| Item | (week) | 54 | 55 | 56 | 57 | 58 | 59 | 60 | 61 |
| Content | 0 | 101.7 | 100.91 | 101.64 | 100.76 | 101.4 | 100.35 | 102.24 | 102.24 |
| | 1 | 100.1 | 99.45 | 99.93 | 97.03 | 99.58 | 99.6 | 98.7 | 98.7 |
| | 2 | 96.93 | 98.18 | 94.64 | 91.15 | 98.04 | 100.5 | 99.26 | 99.26 |
| Size | 0 | 23.19 | 32.44 | 37.8 | 25.04 | 41.85 | 38.38 | 48.13 | 36.88 |
| | 1 | 23.55 | 34.54 | 46.91 | 31.81 | 42.39 | 35.98 | 44.33 | 34.08 |
| | 2 | 27.79 | 35.31 | 51.53 | 54.98 | 42.5 | 36.27 | 44.19 | 38.62 |

As shown in Table 8, the content of cyclosporine in Examples 45 to 61 were all suitable for assay criteria (90-110%) and maintained the initial average particle size of 100 nm or less under high temperature of 70±2° C. even after 2 weeks. The contents of cyclosporine in Examples 45, 51 and 57 wherein the stabilizer was not added were all equal to or greater than 90% and confirmed that the particle size was maintained as 100 nm or less, but in the rest of the Examples wherein the stabilizer was added, it was confirmed that the content of cyclosporine decreasing rate and the particle size variation were very low. Therefore, it was confirmed that the nanoemulsion composition of the present disclosure can maintain its physicochemical stabilities under high temperature, and especially, the physicochemical stabilities could be further improved when the stabilizer is added.

### Experimental Example 5

### Evaluation of Stability of the Nanoemulsion Composition

5.1 Evaluation of Long-Term Stability of the Nanoemulsion Composition

In order to use as the ophthalmic composition, long-term physicochemical stability should be secured under accelerated storage condition (40±2° C., 25% RH or less) which is a condition that can confirm the stability of sample as a 6-month short-term stability data for authorization before long-term physicochemical stability test under room temperature (25±2° C., 40±5% RH) and 24 to 36 months. Therefore, the nanoemulsion compositions of Examples 45 to 61 were stored for 6 months under room temperature and the accelerated storage condition, and the content of cyclosporine and particle size of the nanoemulsion composition were measured at 0^{th}, 3^{rd} and 6^{th} month. A method of measuring content of cyclosporine and particle size was equivalent to Experimental Example 3.

The results of the above are shown in Tables 9 and 10 below.

As shown in Tables 9 and 10, the nanoemulsion compositions of Examples 45 to 61 were confirmed as very stable under the room temperature condition in Table 9 and the accelerated condition in Table 10 due to low variation of content and particle size in long-term.

TABLE 9

| | Duration | Example | | | | | | | | |
|---|---|---|---|---|---|---|---|---|---|---|
| Item | (month) | 45 | 46 | 47 | 48 | 49 | 50 | 51 | 52 | 53 |
| Content | 0 | 101.97 | 102.0 | 101.66 | 98.32 | 99.91 | 98.32 | 101.87 | 100.6 | 100.36 |
| | 3 | 96.08 | 103.43 | 101.37 | 99.45 | 100.63 | 100.56 | 96.24 | 102.57 | 101.36 |
| | 6 | 96.33 | 103.26 | 101.49 | 101.85 | 104.24 | 103.62 | 98.84 | 104.22 | 102.57 |
| Size | 0 | 17.85 | 30.59 | 25.41 | 24.06 | 33.15 | 36.11 | 18.53 | 32.64 | 30.14 |
| | 3 | 21.08 | 31.44 | 26.97 | 24.24 | 35.44 | 45.79 | 18.62 | 32.96 | 31.20 |
| | 6 | 20.88 | 31.14 | 26.52 | 24.20 | 35.25 | 45.66 | 18.63 | 33.05 | 31.20 |

| | Duration | Example | | | | | | | |
|---|---|---|---|---|---|---|---|---|---|
| Item | (month) | 54 | 55 | 56 | 57 | 58 | 59 | 60 | 61 |
| Content | 0 | 101.7 | 100.91 | 101.64 | 100.76 | 101.4 | 100.35 | 102.24 | 102.24 |
| | 3 | 100.64 | 101.17 | 102.74 | 96.07 | 103.89 | 103.60 | 104.26 | 102.68 |
| | 6 | 101.08 | 104.85 | 102.23 | 99.37 | 103.51 | 104.58 | 105.97 | 100.18 |
| Size | 0 | 23.19 | 32.44 | 37.8 | 25.04 | 41.85 | 38.38 | 48.13 | 36.88 |
| | 3 | 25.77 | 32.75 | 44.30 | 19.19 | 41.90 | 50.55 | 36.68 | 39.26 |
| | 6 | 22.16 | 32.36 | 44.46 | 19.64 | 48.30 | 52.81 | 36.47 | 41.18 |

TABLE 10

| | Duration | Example | | | | | | | | |
|---|---|---|---|---|---|---|---|---|---|---|
| Item | (month) | 45 | 46 | 47 | 48 | 49 | 50 | 51 | 52 | 53 |
| Content | 0 | 101.97 | 102.0 | 101.66 | 98.32 | 99.91 | 98.32 | 101.87 | 100.6 | 100.36 |
| | 3 | 102.27 | 99.79 | 100.47 | 101.39 | 103.49 | 101.97 | 105.02 | 96.70 | 100.28 |
| | 6 | 102.86 | 100.87 | 102.21 | 102.01 | 101.95 | 102.31 | 102.01 | 102.54 | 101.87 |
| Size | 0 | 17.85 | 30.59 | 25.41 | 24.06 | 33.15 | 36.11 | 18.53 | 32.64 | 30.14 |
| | 3 | 31.99 | 41.36 | 41.45 | 34.02 | 54.54 | 74.07 | 29.10 | 48.86 | 47.68 |
| | 6 | 31.47 | 40.70 | 40.88 | 33.80 | 54.79 | 73.64 | 29.20 | 49.81 | 47.56 |

| | Duration | Example | | | | | | | |
|---|---|---|---|---|---|---|---|---|---|
| Item | (month) | 54 | 55 | 56 | 57 | 58 | 59 | 60 | 61 |
| Content | 0 | 101.7 | 100.91 | 101.64 | 100.76 | 101.4 | 100.35 | 102.24 | 102.24 |
| | 3 | 99.10 | 105.87 | 106.93 | 106.18 | 99.27 | 100.60 | 104.49 | 104.62 |
| | 6 | 99.95 | 102.18 | 103.35 | 107.29 | 102.39 | 104.02 | 103.43 | 99.84 |
| Size | 0 | 23.19 | 32.44 | 37.8 | 25.04 | 41.85 | 38.38 | 48.13 | 36.88 |
| | 3 | 31.80 | 60.35 | 71.25 | 23.77 | 44.96 | 58.16 | 41.02 | 40.46 |
| | 6 | 31.62 | 58.92 | 71.62 | 23.78 | 45.39 | 55.42 | 41.15 | 43.02 |

### 5.2 Evaluation of a Distribution Stability of the Nanoemulsion Composition

In order to evaluate the long-term stability of the nanoemulsion composition compare to an emulsion and a suspension, dispersion stability test was conducted by using the Turbiscan (Turbiscan Ageing Station, Formulaction, France) in accordance with the manufacturer's manual. The constitutions of the nanoemulsion composition, the emulsion and the suspension used in the test were shown in Table 11 below and the nanoemulsion composition were prepared by using the same method to Experimental Example 4(1). The preparation method of the emulsion follows: The stabilizer and the isotonizing agent of Table 11 were hydrated in the aqueous solvent and its pH was adjusted to 7.2 by using NaOH and HCl. The oily phase was prepared by completely dissolving cyclosporine, the nonaqueous solvent and the emulsifier in content stated in Table 11 via the same method to Experimental Example 1. The prepared oily phase was put in the aqueous solvent and stirred under 400-500 rpm and room temperature by using the stirrer (Super-Nuova™ Multi-place, Thermo Scientific). It was emulsified by using the high speed stirrer (Homomixer T-Basic 25, IKA™) under speed range of 9000-17500 rpm, and the emulsion was prepared through a bubble removal process and a cooling process to a room temperature. The suspension of this example was prepared through following methods: The stabilizer and the isotonizing agent of Table 11 were hydrated in the aqueous solvent and its pH was adjusted to 7.2 by using NaOH and HCl. Cyclosporine and the emulsifier in content stated in Table 11 were put into the aqueous solvent and stirred under 400-500 rpm and room temperature by using the stirrer (Super-Nuova™ Multi-place, Thermo Scientific) for 10 minutes. It was dispersed by using the high speed stirrer (Homomixer T-Basic 25, IKA™) under speed range of 9000-13500 rpm, and the suspension was prepared through the bubble removal process and the cooling process to a room temperature.

TABLE 11

| Ingredient | | Example | | Emul- | Sus- |
|---|---|---|---|---|---|
| (w/v %) | | 62 | 63 | sion | pension |
| Active | Cyclosporine | 0.05 | 0.05 | 0.05 | 0.05 |
| ingredient | | | | | |
| Non- | Castor Oil | 0.42 | 0.42 | 1.26 | — |
| aqueous | | | | | |
| Solvent | | | | | |
| Emulsifier | Cremophor | 1.8 | 1.8 | — | — |
| | ELP | | | | |
| | Polysorbate | — | — | 1.0 | 1.0 |
| | 80 | | | | |
| | polyethylene | 0.4 | 0.4 | — | — |
| | glycol 400 | | | | |
| | Propylene | 0.3 | 0.3 | — | — |
| | Glycol | | | | |
| Stabilizer | Sodium Car- | 0.1 | 1.0 | — | 0.5 |
| | boxymethyl | | | | |
| | Cellulose | | | | |
| | Xanthan | 0.1 | 0.6 | 0.6 | 0.3 |
| | Gum | | | | |
| Buffering | Boric Acid | 0.2 | 1.0 | — | — |
| Agent | | | | | |
| Isotonizing | Glycerol | 1.7 | 0.3 | 2.2 | 2.2 |
| Agent | | | | | |
| pH | NaOH | q.s → | q.s → | q.s → | q.s → |
| adjusting | | 7.2 | 7.2 | 7.2 | 7.2 |
| agent | | | | | |
| Aqueous | Purified | q.s | q.s | q.s | q.s |
| Solvent | Water(ml) | | | | |

The variation of the dispersion stability according to time under certain temperature can be measured when using the Turbiscan, and thus the dispersion stability was measured at 50° C. in conformity with the manufacturer's manual. In detail, the nanoemulsion composition, the emulsion and the suspension were shaken and injected to the Turbiscan, respectively, it was rested under temperature of 50° C. for 48 hours, and variation pattern of each sample according to obtained Turbiscan Stability Index (TSI) which was measured by the Turbiscan was observed.

TSI results of Examples 62 and 63 are illustrated in FIG. 2.

As illustrated in FIG. 2, TSI value of the nanoemulsion composition after 48 hours were 9.2 (Example 62) and 10.6 (Example 63). These TSI values were very low compared to that of the emulsion (i.e. 62.4) and the suspension (i.e. 93.8) which were measured under the same conditions. Accordingly, it was confirmed that the stability of the prepared nanoemulsion composition was markedly superior to the existing emulsions or suspensions.

### Experimental Example 6

### In Vitro Drug Release Test of the Nanoemulsion Composition

To confirm whether the prepared nanoemulsion composition releases cyclosporine A (i.e. active ingredient) with an appropriate rate, in vitro cellulose membrane-release assay which evaluates release of drugs was conducted. Constitutions of the nanoemulsion composition for this assay was identical to that of Examples 62 and 63 in Experimental Example 5(2), and prepared by the same method to Experimental Example 4(1). The commercially available Restasis™ was used as a control drug.

In detail, the membrane (100 kDa Cellulose Ester Membrane) was cut to the appropriate size and soaked over an hour in a medium solution (70% MeOH+30% Balanced Salt Solution (BSS)). The prepared medium in beakers of the same size was dispensed as 100 ml, and magnetic bars in the same shape and size were placed in each beaker. The opposite ends of the membranes were folded and sealed with sealing bars, and the nanoemulsion composition and the control drug were put inside of the membranes respectively, and inserted the membranes containing the drug into the beakers at the same height while the membranes were completely immersed in the beakers but not to contact with the magnetic bars. Stirring was conducted at the same time under identical speed of 150 rpm. The samples were obtained at appropriate time intervals and measured the amount of cyclosporine by the content test through the UPLC in the same manner to Experimental Example 4(2).

As a result of the drug release test, it was confirmed that the nanoemulsion compositions of Examples 62 and 63 commonly maintained constant drug concentration after about 5 hours from the start of the test and reached to Steady State Concentration (Css). Accordingly, the endpoint of the drug release test was set at 6 hours after the start of the test. The concentration of the drug in Example 62 was confirmed as 52.0% through measurement after 5 hours, and it was shown as 54.2% after 6 hours (i.e. the endpoint). These drug concentration was very similar to 55.3% (5 hours) and 56.3% (6 hours) which were the concentration of the control drug (i.e. Restasis™), and thus it was confirmed that the prepared nanoemulsion composition was a suitable composition for eye drop.

### Experimental Example 7

### Evaluation of Ocular Irritation of the Nanoemulsion Composition

Ocular irritation evaluation was conducted for testing sensation by ocular instillation by using the composition of Example 62 within the prepared nanoemulsion compositions. 30 μl of the nanoemulsion composition of Example 62 was administered to both eyes of 40 healthy adults, and burning sensation and foreign body sensation of drop into the eyes of each person were evaluated and scored after 10 minutes according to the scales in Table 12 below. Restasis™ was used as the control drug.

TABLE 12

| Scale | Burning Sensation | Foreign Body Sensation |
|---|---|---|
| 0 | No pruritus, very soft | No foreign body sensation, |
| | | no viscous sensation |
| 1-2 | Little pruritus(stinging) | Little foreign body |
| | | sensation and viscosity |
| 3-4 | Pruritus(stinging) | Continuing foreign body |
| | | sensation accompanying |
| | | little pain, strong |
| | | sensation of viscosity |
| 5 | Immediate sensation of irritation | Long-lasting foreign |
| | and very uncomfortable | body sensation and |
| | | viscosity with strong pain |

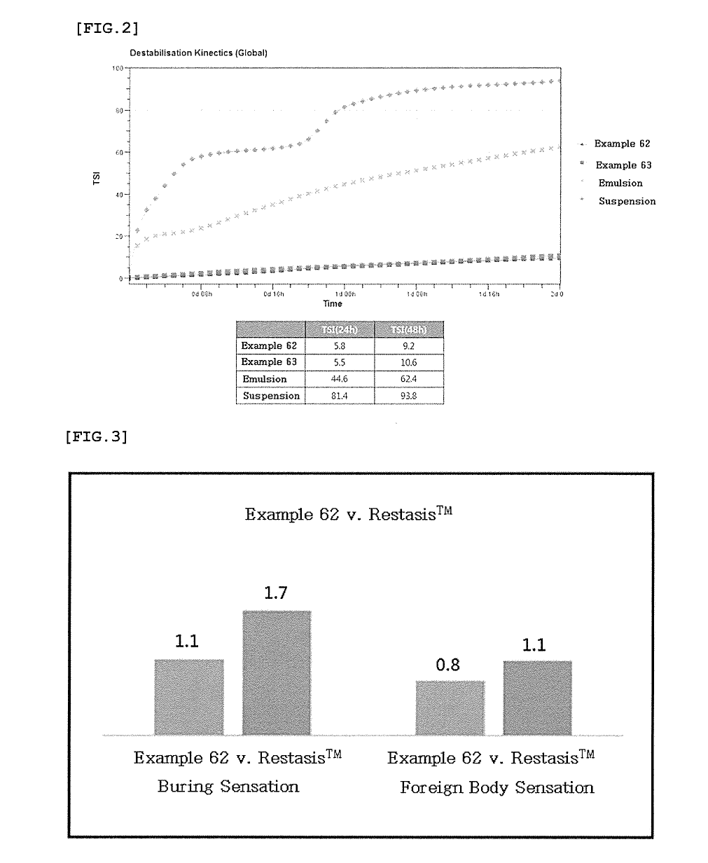

The results were shown in Table 13 and FIG. 3.

TABLE 13

| | Burning Sensation | | Foreign Body Sensation | |
|---|---|---|---|---|
| Subject | Example 62 | Restasis ™ | Example 62 | Restasis ™ |
| 1 | 0 | 4 | 0 | 4 |
| 2 | 1 | 3 | 1 | 3 |
| 3 | 1 | 0 | 1 | 0 |
| 4 | 2 | 2 | 0 | 2 |
| 5 | 2 | 0 | 1 | 0 |
| 6 | 2 | 4 | 1 | 3 |
| 7 | 3 | 2 | 0 | 0 |
| 8 | 2 | 0 | 1.5 | 1 |
| 9 | 2 | 1 | 0 | 0 |
| 10 | 1 | 0 | 0 | 0 |
| 11 | 0 | 0 | 1 | 1 |
| 12 | 1 | 3 | 0 | 0 |
| 13 | 0 | 2 | 0 | 0 |
| 14 | 0 | 1 | 0 | 0 |
| 15 | 2 | 1 | 0 | 2 |
| 16 | 0 | 0 | 2 | 2 |
| 17 | 2 | 1 | 1 | 1 |
| 18 | 0 | 3 | 0 | 0 |
| 19 | 1 | 3 | 1 | 2 |
| 20 | 0 | 0 | 0 | 0 |
| 21 | 3 | 2 | 3 | 1 |
| 22 | 1 | 3 | 3 | 1 |
| 23 | 1 | 0 | 2 | 0 |
| 24 | 0 | 3 | 0 | 0 |
| 25 | 0 | 1 | 0 | 0 |
| 26 | 1 | 4 | 0 | 4 |
| 27 | 0 | 0 | 2 | 1 |
| 28 | 3 | 4 | 1 | 1 |
| 29 | 0 | 0 | 1 | 0 |
| 30 | 0 | 2 | 0 | 2 |
| 31 | 4.5 | 3.5 | 3 | 3 |
| 32 | 0 | 1 | 0 | 1 |
| 33 | 0 | 1 | 0 | 0 |
| 34 | 3 | 4 | 2 | 1 |
| 35 | 0 | 2 | 1 | 1 |
| 36 | 1 | 2 | 0 | 1 |
| 37 | 3 | 2 | 0 | 1 |
| 38 | 0 | 2 | 1 | 1 |
| 39 | 0 | 1 | 0 | 1 |
| 40 | 0 | 2 | 1 | 1 |
| Total | 42.5 | 69.5 | 30.5 | 42 |
| Average | 1.1 | 1.7 | 0.8 | 1.1 |

As shown in Table 13 and FIG. 3, the ophthalmic nanoemulsion of Example 62 exhibited average scores 1.1 of burning sensation and 0.8 of foreign body sensation which were lower than that of control drug Restasis™ (i.e., 1.7 of burning sensation and 1.1 of foreign body sensation). Namely, it was confirmed that the ophthalmic nanoemulsion was a composition that shows more improved sensation by ocular instillation compared to the existing Restasis™.

### INDUSTRIAL APPLICABILITY

The ophthalmic nanoemulsion composition according to the present disclosure is characterized in having average particle size of is 200 nm or less, preferably 100 nm or less and having a narrow particle distribution, and thus sterilizing filtration is available, stability can be improved and has superior effect in clinical enhancement of foreign body sensation and visual disturbance. Therefore, the ophthalmic nanoemulsion composition of the present disclosure can be effectively used as an ophthalmic composition.

1. (canceled)

2. An ophthalmic nanoemulsion composition, comprising:
cyclosporine in the amount of 0.02 to 0.3 w/v % based on the total amount of the composition;
one or more nonaqueous solvents in the amount of 0.1 to 2.5 w/v % based on the total amount of the composition selected from the group consisting of vegetable oils, C₁₄-C₂₀ fatty acid esters and C₆-C₁₂ fatty acid esters of glycerol;
one or more hydrophilic emulsifiers in the amount of 0.1 to 5.0 w/v % based on the total amount of the composition selected from the group consisting of polyoxyethylene hydrogenated castor oils, polyoxyethylene sorbitan fatty acid esters, polyoxyethylene fatty acid esters and polyoxyl 35 castor oil;
one or more hydrophobic emulsifiers in the amount of 0.1 to 5.0 w/v % based on the total amount of the composition selected from the group consisting of sorbitan fatty acid esters, glycerin fatty acid esters, diethylene glycol monoethyl ether, polyethylene glycols, propylene glycol and propylene glycol esters of fatty acids; and
an aqueous solvent,
wherein the composition has an average particle size range of 1 nm to 100 nm.

3. The ophthalmic nanoemulsion composition of claim 2, wherein the nonaqueous solvent is at least one selected from the group consisting of castor oil, labrafac, miglyol 812, ethyl oleate and isopropyl myristate.

4. The ophthalmic nanoemulsion composition of claim 3, wherein the nonaqueous solvent is the castor oil.

5. The ophthalmic nanoemulsion composition of claim 2, wherein the hydrophilic emulsifier is a polyoxyethylene hydrogenated castor oil, polyoxyl 35 castor oil or a polyoxyethylene sorbitan fatty acid ester.

6. The ophthalmic nanoemulsion composition of claim 2, wherein the hydrophobic emulsifier is at least one selected from the group consisting of the polyethylene glycol, the propylene glycol and the diethylene glycol monoethyl ether.

7. The ophthalmic nanoemulsion composition of claim 2, wherein the ophthalmic nanoemulsion composition further comprises one or more stabilizers selected from the group consisting of carboxymethyl cellulose (CMC), hydroxypropyl methylcellulose (HPMC), hydroxyethylcellulose (HEC), polyvinyl alcohol (PVA), polyvinylpyrrolidone (PVP), carbomer, gellan gum, xanthan gum, hyaluronic acid (HA), sodium hyaluronate, sodium alginate and dextran.

8. The ophthalmic nanoemulsion composition of claim 7, wherein the amount of the stabilizer is 0.01-2.0 w/v % based on the total amount of the composition.

9. The ophthalmic nanoemulsion composition of claim 2, wherein a maximum particle size of the ophthalmic nanoemulsion composition is equal to or less than 220 nm.

10. An ophthalmic nanoemulsion composition, comprising:
cyclosporine in the amount of 0.02-0.3 w/v % based on the total amount of the composition;
castor oil in the amount of 8 times or more of the amount of the cyclosporine to 2.5 w/v % or less based on the total amount of the composition;
one or more hydrophilic emulsifiers selected from the group consisting of polyoxyethylene hydrogenated castor oils, polyoxyethylene sorbitan fatty acid esters, polyoxyl 35 castor oil and polyoxyethylene fatty acid esters;
one or more hydrophobic emulsifiers selected from the group consisting of sorbitan fatty acid esters, glycerin fatty acid esters, diethylene glycol monoethyl ether, polyethylene glycols, propylene glycol and propylene glycol esters of fatty acids; and
an aqueous solvent.

11. The ophthalmic nanoemulsion composition of claim 10, wherein:
the hydrophilic emulsifier is polyoxyl 35 castor oil; and
the amount of hydrophilic emulsifier is 1.6 times or more of the amount of the castor oil to 5.0 w/v % or less based on the total amount of the composition.

12. The ophthalmic nanoemulsion composition of claim 11, wherein:
the hydrophobic emulsifier is at least one selected from the group consisting of polyethylene glycol, propylene glycol and diethylene glycol monoethyl ether; and
the amount of the hydrophobic emulsifier is 0.1 w/v % or more based on the total amount of the composition to 3 times or less based on the amount of the hydrophilic emulsifier.

13. The ophthalmic nanoemulsion composition of claim 10, wherein:
the hydrophilic emulsifier is polyoxyl 35 castor oil and the amount of the hydrophilic emulsifier is 12.8 times or more of the amount of the cyclosporine to 5.0 w/v % based on the total amount of the composition; and
the hydrophobic emulsifier is at least one selected from the group consisting of polyethylene glycol, propylene glycol and diethylene glycol monoethyl ether, and the amount of the hydrophobic emulsifier is 0.1 w/v % to 5.0 w/v % based on the total amount of the composition.

14.-15. (canceled)

16. A method of preventing or treating an ophthalmic disease, comprising administering into an eye of a patient the ophthalmic nanoemulsion composition of claim 2.

17. The method of of claim 16, wherein the ophthalmic disease is Sjogren syndrome or dry eye syndrome.

18. The composition of claim 2, wherein:
the nonaqueous solvent is present in the amount of 8 times or more of the amount of the cyclosporine to 2.5 w/v % or less based on the total amount of the composition and is castor oil;
the hydrophilic emulsifier is present in the amount of 1.6 times or more of the amount of the castor oil to 5.0 w/v % or less based on the total amount of the composition and is the polyoxyl 35 castor oil; and
the hydrophobic emulsifier is present in the amount of 0.1 to 5.0 w/v % based on the total amount of the composition and is at least one selected from the group consisting of the polyethylene glycols, the propylene glycol and the diethylene glycol monoethyl ether.
